(19) United States
(12) Patent Application Publication
(10) Pub. No.: US 2021/0401347 A1
(43) Pub. Date: Dec. 30, 2021

(54) MACHINE-LEARNING MODELS FOR ECG-BASED TROPONIN LEVEL DETECTION

(71) Applicant: Mayo Foundation for Medical Education and Research, Rochester, MN (US)

(72) Inventors: Paul A. Friedman, Rochester, MN (US); Itzhak Zachi Attia, Rochester, MN (US); Allan S. Jaffe, Rochester, MN (US); Suraj Kapa, Rochester, MN (US); Francisco Lopez-Jimenez, Rochester, MN (US); Yader B. Sandoval Pichardo, La Crosse, WI (US)

(21) Appl. No.: 17/357,735

(22) Filed: Jun. 24, 2021

Related U.S. Application Data
(60) Provisional application No. 63/043,744, filed on Jun. 24, 2020.

(51) Int. Cl. A61B 5/346; G06N 3/08; G16H 50/20; G16H 50/70; G16H 40/63; G16H 10/60; A61B 5/145

(52) U.S. Cl. CPC A61B 5/346; G06N 3/08; G16H 50/20; A61B 5/14546; G16H 40/63; G16H 10/60; G16H 50/70

(57) ABSTRACT

Systems and methods for assessing the condition of a heart of an individual. A system obtains electrocardiogram (ECG) data that describes a result of an ECG of the individual. The ECG data can be provided to a machine-learning model, which processes the data and generates an output indicative of the condition of the heart. The output relates to a level of troponin in a bloodstream of the individual. The system can then provide the output of the machine-learning model to a post-processing resource.

### CROSS-REFERENCE TO RELATED APPLICATIONS

This application claims the benefit of priority to U.S. Provisional Application Ser. No. 63/043,744, which was filed on Jun. 24, 2020. The disclosure of the prior application is considered part of and is incorporated by reference in the disclosure of this application.

### BACKGROUND

### 1. Technical Field

This specification relates to electrocardiograms (ECGs), and the use of neural networks or other machine-learning models to process ECG data and estimate troponin levels indicative of heart injury.

### 2. Background Information

Damage to the heart causes it to release troponin into the bloodstream. Troponin levels in the blood are normally very low, but injury to myocytes causes blood levels to increase significantly.

Neural networks are machine-learning models that employ multiple layers of operations to predict one or more outputs from one or more inputs. Neural networks typically include one or more hidden layers situated between an input layer and an output layer. The output of each layer is used as input to another layer in the network, e.g., the next hidden layer or the output layer.

Each layer of a neural network specifies one or more transformations to be performed on input to the layer. Some neural network layers have operations that are referred to as neurons, which implement transformations according to weights established during a training process. Each neuron can receive one or more inputs and generate an output for another neural network layer. The transformations of each layer can be carried out by one or more computers at one or more locations having installed software modules that implement the transformations.

### SUMMARY

This specification describes systems, methods, devices, and other techniques for assessing the condition of a heart in a mammal based on troponin levels. The disclosed techniques can advantageously enable non-invasive estimation of troponin levels, or non-invasive detection or prediction of change in troponin levels. In this way, assessments may be made based on measurements acquired from a patient's home, and can, in some examples, serve as an alert to the presence of cardiac injury. For instance, such assessments could reassure a person that symptoms are not associated with heart injury (at home, or in the emergency department, clinic, ward, or other environment) or that heart injury (including myocardial infarction) is present, and immediate action is warranted. Such a painless, point of care, under one minute, non-invasive, bloodless test can be immensely useful in clinical practice.

### BRIEF DESCRIPTION OF DRAWINGS

FIG. 1 is a flowchart of an example process for estimating/predicting a level of troponin in the bloodstream of a mammal using machine-learning models processing electrocardiogram data.

### DETAILED DESCRIPTION

One or more neural networks can be employed to predict a person's troponin levels based on processing data characterizing an ECG signal of a short duration (e.g., 1 minute or less). The ECG signal can reflect a change in patient troponin level or its absolute value, or its value relative to a threshold. The ECG signal may reflect a single lead, or multiple leads. It may be acquired from surface, subcutaneous, or intracardiac electrodes. Surface electrodes may be integrated into wearables or other devices.

In some implementations, the system uses the neural network to evaluate whether the troponin levels are above or below the population on a percentile-basis adjusted by sex (e.g., 99%).

In some implementations, the system uses the neural network to estimate a numerical troponin level. The estimate can relate to a recent, current, or future level of troponin in the patient's bloodstream.

In some implementations, the system uses the neural network to predict whether future troponin levels in the patient's bloodstream (e.g., in 2-6 hours) will be the same (no recent injury), higher (showing an active injury) or lower (a recent injury).

In some implementations, the system uses the neural network to evaluate who does not have an injury and would not benefit from further workup or immediate evaluation.

The ECG signal can be acquired using one or two leads mounted on a phone, a stethoscope, or other ECG-enabled device (e.g., wearables), few leads from a 12 lead device or any number of leads.

A machine learning model utilizing a convolutional, recurrent, or other neural network structure can be employed using selected data with ECGs and troponin levels. This may admixed with human feature selection, vector machines, and hidden Markov models to optimize performance.

Suitable machine-learning algorithms may be applied to train the neural network, such as backpropagation with gradient descent. The model can be trained, for example, on data from patients who have pairs of fifth generation troponin levels and ECG. Different time intervals between the ECG and troponin levels can be used based on whether the model is trained to estimate current troponin levels or predict future troponin levels or conditions. For example, training samples for estimation of current troponin levels may include a training input representing an ECG of a patient acquired in close temporal proximity to a blood test of the patient in which troponin levels were quantified from an assay of the blood. The troponin level from the assay can be used as the target. In other examples, training samples for predicted troponin levels may include a training input representing an ECG of a patient and the measured troponin level at some time in the future (e.g., 2-6 hours).

In some aspects, computer-based systems are provided for assessing the condition of a heart of an individual. A system obtains electrocardiogram (ECG) data that describes a result of an ECG of the individual. The ECG data can be provided to a machine-learning model, which processes the data and generates an output indicative of the condition of the heart. The output relates to a level of troponin in a bloodstream of the individual. The system can then provide the output of the machine-learning model to a post-processing resource.

These and other implementations can further include one or more of the following features.

The neural network can be a convolutional neural network, a feedforward neural network, or a recurrent neural network.

The neural network can include at least one of convolutional or recurrent layers.

The ECG data can be acquired using a twelve-lead ECG, a subset of leads from a twelve-lead ECG, or a single-lead ECG. Acquiring the ECG can include detecting electrical activity of the mammal from electrodes communicably coupled to a smartphone, a tablet computing device, a notebook computer, a desktop computer, or a wearable computing device.

Providing the output of the machine-learning model to a post-processing resource can include at least one of storing the output in a memory of a computer, providing an indication of the output for presentation to a user on an electronic display, generating an alert for a user based on the output, or generating an entry in a medical record of the mammal based on the output. The user can be the mammal, an agent of the mammal, or a healthcare provider associated with the mammal.

The output can indicate whether the level of troponin is greater than a threshold level. The threshold level can be based on a level exhibited by a pre-defined percentile of a population. The population can be limited to individuals of a particular sex.

The output can be a numerical estimation of a current level of troponin in the bloodstream.

The output can include a prediction of whether (i) a future level of troponin in the bloodstream of the mammal will remain unchanged from a current level of troponin in the bloodstream, (ii) the future level of troponin will be lower than the current level of troponin in the bloodstream of the mammal, or (iii) the future level of troponin will be higher than the current level of troponin in the bloodstream of the mammal.

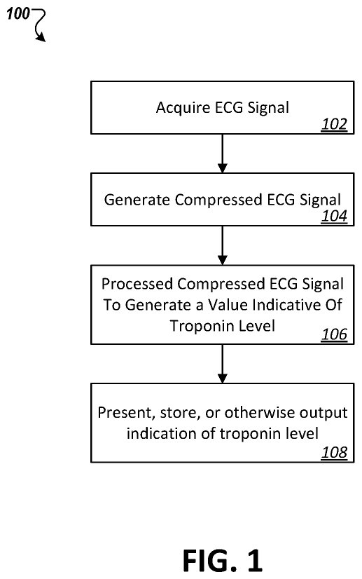

FIG. 1 is a flowchart of an example process 100 for estimating/predicting a level of troponin in a patient's bloodstream by processing ECG data (e.g., data describing an electrocardiogram signal from one or more leads) using machine-learning models. An ECG signal is acquired (102). The ECG data can be acquired using a twelve-lead ECG, a subset of leads from a twelve-lead ECG, or a single-lead ECG. Acquiring the ECG can include detecting electrical activity of the mammal from electrodes communicably coupled to a smartphone, a tablet computing device, a notebook computer, a desktop computer, or a wearable computing device. The ECG signal can be recorded from the patient for a first time duration (e.g., at least 1 minute, 2 minutes, 5 minutes, 10 minutes, 15 minutes, 20 minutes, 30 minutes, 45 minutes, 1 hour or more). The ECG signal can be sampled and quantized, and then compressed to generate compressed ECG data describing a compressed version of the ECG signal (104). It is preferable to compress the ECG data in many cases in order to increase the computational efficiency of estimating/predicting troponin levels, to decrease the size of the machine-learning model (e.g., reduce the number of parameters, nodes, and/or layers in a neural network), and to reduce the overall size of the ECG data and model for storage or transmission. In some examples, the ECG data is compressed by reducing the number of leads of ECG data processed by the machine-learning model. For instance, all leads in the original ECG signal can be evaluated against quality criteria and a subset 1, 2, or more of the leads from the original ECG signal can be selected for processing by the machine-learning model. In some examples, the ECG data is compressed by shortening the time duration of the ECG signal processed by the machine-learning model. If the original ECG signal was recorded for a first time duration (e.g., 20 minutes), the compressed ECG signal can be a snippet of the original signal and can have a second time duration (e.g., 5 minutes) that is shorter than the first time duration. This allows the signal to be processed more efficiently since the inputs to the machine-learning model characterize a shorter portion of the ECG signal. In some examples, the level of compression can be adjusted based on one or more characteristics of the patient (e.g., demographic or health characteristics), an indication of computational resources available to perform the prediction/estimation, or quality or other characteristics of the original ECG signal. The compressed ECG signal (or the non-compressed ECG signal if compression was not performed) can be processed to generate a value indicative of an estimated/predicted troponin level (106). The output can be presented, stored, or otherwise provided to one or more users (108).

### Example Implementation #1

Background Myocardial injury results in release of cardiac troponin (cTn) into the bloodstream, readily detected by high-sensitivity cTn (hs-cTn) assays. Since myocyte injury is associated with ECG changes, it was hypothesized that an artificial intelligence ECG (AI-ECG) could identify absence of injury.

Objective. To train and test an AI-ECG convolutional neural network (CNN) to identify patients using a single ECG who are suspected of myocardial infarction who are low risk, with hs-cTn levels below the 99^{th} percentile at test time and for the subsequent 7 hours.

Methods. A CNN tuned to identify the absence of a hs-cTnT (5^{th} Gen cTnT ROCHE DIAGNOSTICS) >15 ng/L for men and >10 ng/L for women was developed. All ECGs were recorded within one hour of the hs-cTnT assay. The study used 73,012 ECGs and hs-cTnT pairs from 47,542 unique patients to train the network, 9031 ECGs from 5,811 patients for internal validation to optimize hyperparameters, and 11,904 ECGs with 21,191 hs-cTnT measurements up to 7 hours after the ECG, from 11,904 different patients as a holdout test set.

Results. The mean age was 63.9±17.5 years, and 30,348 of the 59,446 patients (51%) were male. 5,852 patients (49.1%) had no elevation of hs-cTnT and 6,052 (50.9%) had an hs-cTnT above the 99^{th} percentile at baseline or within 7 hours of the test. Of the 11,904 patients in the test set, using a sensitive threshold, the 12 lead AI ECG identified 1037 patients (8.7%) likely to have a low risk for subsequent hs-cTnT increases >99^{th}(AUC 0.86), and the single lead ECG identified 685 patients. Of the 1037 low risk pts, 59 had an hs-cTnT>99^{th} percentile within 7 hours. The mean maximum hs-cTnT among the 59 low-risk patients was 53 ng/L±92 vs 184 ng/L±1474 in the others. None of these low risk patients died within 14 days of the test.

### Example Implementation #2

Background High-sensitivity cardiac troponin (hs-cTn) assays quantify cTn in patients at very low concentrations. Myocyte injury due to ischemia or other pathologies cause blood levels to increase, which is prognostic. Since myocyte injury is associated with ECG changes, it was hypothesized that an artificial intelligence ECG (AI-ECG) could non-invasively predict current or impending hs-cTnT elevations.

Objective. To develop an AI-ECG convolutional neural network (CNN) to detect an abnormal hs-cTnT (5^{th} Gen cTnT, ROCHE DIAGNOSTICS) concentration using a 12-lead ECG, and a single lead ECG (lead I), which would enable smartphone, home-based detection.

Methods. A single lead and 12-lead ECG CNNs was developed to detect a) hs-cTnT concentrations that were at or above the 6 ng/L limit that can be reported b) above the 99^{th} percentile upper limits of >15 ng/L for men and >10 ng/L for women. All ECGs were recorded within one hour of the hs-cTnT measurements. The study used 73,012 ECG and hs-cTnT pairs from 47,542 unique patients to train the network, 9031 ECGs from 5,811 patients for internal validation to optimize hyperparameters, and 18,276 ECG and hs-cTnT pairs from 11,904 different patients as a holdout test set to determine the area under the receiver-operator curve (AUC).

Results. The mean age was 63.9±17.5 years, and 30,348 of the 59,446 patients (51%) were male. Of the 91,288 hs-cTnT pairs 73,271 (80.2%) were above 6 ng/L and 50,799 (55.6%) are above the 99^{th} percentile. In the test set, the AUC for the detection of a hs-cTnT level higher than 6 ng/L was 0.88 using the 12 lead ECG and 0.834 with the single lead. For the detection of hs-cTnT level above of 99^{th} percentile, the 12 lead ECG AUC was 0.853 and the single lead was 0.806.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, off-the-shelf or custom-made parallel processing subsystems, e.g., a GPU or another kind of special-purpose processing subsystem. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program which may also be referred to or described as a program, software, a software application, an app, a module, a software module, a script, or code) can be written in any form of programming language, including compiled or interpreted languages, or declarative or procedural languages, and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

As used in this specification, an “engine,” or “software engine,” refers to a software implemented input/output system that provides an output that is different from the input. An engine can be an encoded block of functionality, such as a library, a platform, a software development kit (“SDK”), or an object. Each engine can be implemented on any appropriate type of computing device, e.g., servers, mobile phones, tablet computers, notebook computers, music players, e-book readers, laptop or desktop computers, PDAs, smart phones, or other stationary or portable devices, that includes one or more processors and computer readable media. Additionally, two or more of the engines may be implemented on the same computing device, or on different computing devices.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and pointing device, e.g, a mouse, trackball, or a presence sensitive display or other surface by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone, running a messaging application, and receiving responsive messages from the user in return.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain some cases, multitasking and parallel processing may be advantageous.

What is claimed is:

1. A method for assessing a condition of a heart of a mammal, comprising:
obtaining electrocardiogram (ECG) data that describes a result of an ECG of the mammal;
processing, with a machine-learning model, the ECG data to generate an output indicative of the condition of the heart of the mammal, wherein the output relates to a level of troponin in a bloodstream of the mammal; and
providing the output of the machine-learning model to a post-processing resource.

2. The method of claim 1, wherein the neural network is a convolutional neural network, a feedforward neural network, or a recurrent neural network.

3. The method of claim 1, wherein the neural network includes at least one of convolutional or recurrent layers.

4. The method of claim 1, further comprising acquiring the ECG data using a twelve-lead ECG, a subset of leads from a twelve-lead ECG, or a single-lead ECG.

5. The method of claim 4, wherein acquiring the ECG comprises detecting electrical activity of the mammal from electrodes communicably coupled to a smartphone, a tablet computing device, a notebook computer, a desktop computer, or a wearable computing device.

6. The method of claim 1, wherein providing the output of the machine-learning model to a post-processing resource comprises at least one of storing the output in a memory of a computer, providing an indication of the output for presentation to a user on an electronic display, generating an alert for a user based on the output, or generating an entry in a medical record of the mammal based on the output.

7. The method of claim 6, wherein the user is the mammal, an agent of the mammal, or a healthcare provider associated with the mammal.

8. The method of claim 1, wherein the output indicates whether the level of troponin is greater than a threshold level.

9. The method of claim 8, wherein the threshold level is based on a level exhibited by a pre-defined percentile of a population.

10. The method of claim 9, wherein the population is limited to individuals of a particular sex.

11. The method of claim 1, wherein the output is a numerical estimation of a current level of troponin in the bloodstream.

12. The method of claim 1, wherein the output is a prediction of whether (i) a future level of troponin in the bloodstream of the mammal will remain unchanged from a current level of troponin in the bloodstream, (ii) the future level of troponin will be lower than the current level of troponin in the bloodstream of the mammal, or (iii) the future level of troponin will be higher than the current level of troponin in the bloodstream of the mammal.

13. A computing system, comprising:
one or more processors; and
one or more computer-readable media having instructions stored thereon that, when executed by the one or more processors, cause performance of operations comprising:
obtaining electrocardiogram (ECG) data that describes a result of an ECG of the mammal;
processing, with a machine-learning model, the ECG data to generate an output indicative of the condition of the heart of the mammal, wherein the output relates to a level of troponin in a bloodstream of the mammal; and
providing the output of the machine-learning model to a post-processing resource.

14. The computing system of claim 13, wherein the neural network is a convolutional neural network, a feedforward neural network, or a recurrent neural network.

15. The computing system of claim 13, wherein the neural network includes at least one of convolutional or recurrent layers.

16. The computing system of claim 13, wherein the operations comprise acquiring the ECG data using a twelve-lead ECG, a subset of leads from a twelve-lead ECG, or a single-lead ECG.

17. The computing system of claim 16, wherein acquiring the ECG comprises detecting electrical activity of the mammal from electrodes communicably coupled to a smartphone, a tablet computing device, a notebook computer, a desktop computer, or a wearable computing device.

18. The computing system of claim 13, wherein providing the output of the machine-learning model to a post-processing resource comprises at least one of storing the output in a memory of a computer, providing an indication of the output for presentation to a user on an electronic display, generating an alert for a user based on the output, or generating an entry in a medical record of the mammal based on the output.

19. The computing system of claim 18, wherein the user is the mammal, an agent of the mammal, or a healthcare provider associated with the mammal.

20. One or more non-transitory computer-readable media having instructions stored thereon that, when executed by one or more processors, cause performance of operations comprising:
obtaining electrocardiogram (ECG) data that describes a result of an ECG of the mammal;
processing, with a machine-learning model, the ECG data to generate an output indicative of the condition of the heart of the mammal, wherein the output relates to a level of troponin in a bloodstream of the mammal; and
providing the output of the machine-learning model to a post-processing resource.
